(19) United States
(12) Patent Application Publication
(10) Pub. No.: US 2017/0225205 A1
(43) Pub. Date: Aug. 10, 2017

(54) ULTRASONIC PRE-CLEANING BATH AND METHOD

(71) Applicant: Z-PROJECTS BVBA, Massenhoven (BE)

(72) Inventor: Serge WELLENS, Massenhoven (BE)

(73) Assignee: Z-PROJECTS BVBA, Massenhoven (BE)

(21) Appl. No.: 15/218,425

(22) Filed: Jul. 25, 2016

(30) Foreign Application Priority Data
Jun. 1, 2015 (GB) 1509404.8

(51) Int. Cl. B08B 3/12; A61B 90/70

(52) U.S. Cl. CPC B08B 3/12; A61B 90/70

(57) ABSTRACT

An apparatus and method for ultrasonic pre-cleaning cleaning of objects such as medical or surgical devices optionally having a lumen are described of a tank for holding cleaning liquid, the tank having at least one first wall and a bottom and being adapted to hold cleaning liquid and to expose the cleaning liquid to ultrasound; a weir opposite and parallel to the first wall and a first channel adjacent and proximal to the weir and distal with respect to the tank; a first drain located in said first channel for draining cleaning liquid that spills over the weir; a second channel in the form of a depression in the bottom of the tank, and a second drain located in the second channel for draining cleaning liquid from the tank.

The present invention relates to methods and apparatus for cleaning, rinsing or pre-cleaning of objects especially reusable medical or surgical equipment, apparatus or devices, and also cleaning of such equipment or devices which are hollow such as tubular medical or surgical devices with lumens such as endoscopes.

### BACKGROUND OF THE INVENTION

Prior art devices exist for cleaning of medical and surgical devices. Safe reuse of medical devices such as surgical instruments depends upon the effectiveness of the cleansing process. One method of cleaning uses water, for example, having additives such as solvents, detergents or antibacterial and/or antiviral agents, enzymatic detergents alone or in combination with ultrasound. Ultrasound allows rapid cycle times of several minutes. The first step in a cleaning process is usually to rinse off all blood, bodily fluids and tissue as soon as possible after use followed by manual or mechanical cleaning in a washer or ultrasonic bath.

Cleaning the hollow spaces or lumens of medical devices has traditionally required hand operation using a syringe and this can involve flushing the lumen of the device several times. Thus changing from cleaning of handheld small surgical instruments to large devices with lumens involves a lot of manual activity. A typical sequence can be:
a) Clean the devices and instruments in an ultrasound cleaning bath
b) Clean the product with a suitable cleaning brush until all visible residues have been removed from the surface taking special care of products with hidden crevices, lumens or complex geometries,
c) After cleaning, thoroughly rinse several times with a suitable cleaning solution
d) Rinse the product with water
e) Allow to drip dry
f) Clean in a disinfecting solution.
g) Further rinse several times in water
h) Allow water to drip off for a sufficient length of time
i) Dry devices and instruments

Use of brushes increases the risk that “soils” (detritus or debris) are left in the brush and can be transferred to another instrument. In a cleaning bath, detritus and debris released from the medical devices can float to the surface and can be removed by skimming. Detritus and debris that sink to the bottom has to be removed by drains which can get blocked. Every effort needs to be made to prevent anything remaining attached to the devices, even as small as bacteria or viruses. Due to the risk of coagulation the temperature of cleaning baths must be kept below a certain temperature, e.g. 40° C., and this means that the amount of ultrasound energy that can be injected into the cleaning liquid is limited as such energy will raise the temperature of the cleaning liquid. On the other hand, introducing more sonic energy from several sides can produce a more thorough cleaning and can avoid acoustic shadows.

At the end of the cleaning process the devices or instruments should not show any visual contamination and the total amount of residual proteins on and in the devices or instruments should be (EN ISO 15883-1:2006, C2) less than 100 μg (warning level) or 200 μg (threshold) or in some countries less than 6.4 μg/cm². Usually effective cleaning requires a sequence of cleaning processes, e.g. a series of tanks and/or washing machines. The overall safety and efficacy of the complete cleaning process can be improved if the early processes such as rinsing and pre-cleaning do a maximum to remove unwanted material usually called “soils”.

### SUMMARY OF THE INVENTION

It is an object of the present invention to provide a good ultrasonic pre-cleaning or cleaning apparatus and method.

Advantages of the apparatus and method according to embodiments of the present invention can be one or more:
economical;
solves one or more of the problems encountered with prior art apparatus such as, for example, the cleaning of reusable medical and/or surgical devices, in particular tubular items such as endoscopes;
insertion of high density ultrasound while controlling temperature of the cleaning liquid;
avoidance of dead spaces and eddies.

One aspect the present invention is an apparatus for ultrasonic pre-cleaning or cleaning of objects such as medical or surgical devices optionally having a lumen, the apparatus comprising:

a tank having a tank volume for holding cleaning liquid, the tank having at least one first wall and a bottom and being adapted to hold cleaning liquid and to expose the cleaning liquid when in the tank to ultrasound;
a weir facing (e.g. parallel to) the at least one first wall;
a first channel adjacent and proximal to the weir and distal with respect to the tank volume;
a first drain located in said first channel for draining cleaning liquid that spills over the weir;
a second channel in the form of a depression in the bottom of the tank, and
a second drain located in the second channel for draining cleaning liquid from the tank.

In this aspect of the present invention an apparatus is provided for ultrasonic pre-cleaning or cleaning whereby the weir is in a topological relationship to the at least one wall such that a surface liquid flow initiated at the at least one wall traverses the complete surface of the cleaning liquid and reaches the weir, whereby each part of the surface liquid flow initiated at the at least one wall maps to a part of the weir such that no dead spaces are created within the surface liquid flow.

In addition, in this aspect of the present invention an apparatus is provided for ultrasonic cleaning whereby the second channel is in a topological relationship to the at least one wall and/or the weir and/or at least one second wall such that a bottom liquid flow initiated at the level of the bottom of the tank traverses the complete bottom of the tank liquid and reaches the second channel, whereby each part of the bottom flow maps to a part of the second channel such that no dead spaces are created within the bottom flow.

In this aspect of the present invention a brushless apparatus for ultrasonic cleaning of objects such as medical or surgical devices optionally having a lumen, is provided.

In another aspect of the present invention a console cleaning system can be provided that integrates one or more ultrasonic cleaning tanks, with other tanks such as rinse tanks, or washing machines or a dryer for batch cleaning. In accordance with another aspect of the present invention such as systems is automated through the use of a PLC.

Ultrasonic transducers are provided for inserting ultrasonic energy through the bottom of the tank and through the first and second walls. By using several walls the ultrasound energy in the cleaning liquid can be high. Also ultrasound shadows can be avoided.

An outlet can be provided for delivering cleaning liquid and/or steam for the cleaning of objects with a lumen.

A first skimmer can be provided for moving floating debris or detritus towards and over the weir. This first skimmer is preferably adapted to insert cleaning liquid through the at least one first or second wall for moving floating debris or detritus towards and over the weir. The flow is preferably of a laminar nature.

The first skimmer can comprise a first line of holes, or a slit or slits located in the at least one first or at least one second wall for inserting liquid through the at least one first or second wall.

Also a second skimmer can be provided for moving sunken debris or detritus along the bottom of the tank towards the second channel and down the second drain. This second skimmer can be adapted to insert cleaning liquid through the at least one first or second wall for moving sunken debris or detritus towards the second channel and down the second drain. The second skimmer can comprise a second line of holes, or a slit or slits located in the at least one first or second wall for inserting liquid through the at least one first or second wall. The flow is preferably of a laminar nature.

Preferably the cleaning liquid in the tank is exposed to 20 to 40 watts per litre, or 30 to 35 watts per litre of ultrasound energy. This allows for a rapid and thorough cleaning which lowers the requirements for downstream washing machines and contributes to a higher efficacy of the overall process.

The present invention also provides a method of ultrasonic pre-cleaning or cleaning of objects such as medical or surgical devices optionally having a lumen, in a tank holding cleaning liquid, the tank having at least one first wall and a bottom and being adapted to hold cleaning liquid and to expose the cleaning liquid in the tank to ultrasound;

a weir opposite (e.g. parallel) to the at least one first wall and a first channel adjacent and proximal to the weir and distal with respect to the tank; and
ultrasonic transponders for injecting ultrasonic energy into the tank,
the method comprising:
removing floating detritus or debris by a first directing of cleaning liquid to spill over the weir and draining the floating detritus or debris and cleaning liquid from the tank, and
removing sunken detritus or debris by a second directing of cleaning liquid to flow along the bottom of the tank and draining the sunken detritus or debris and cleaning liquid from the tank.

The present invention also provides a control system for a console pre-cleaning or cleaning system comprising a main controller to control the operation of the following optional control elements:
A source of cleaning liquid;
A pump and valve system to provide liquid to rinsing tank;
A chemical dispensing system for dosing chemicals (optional) to the liquid supplied to the rinsing tank;
A pump and valve system to provide liquid to cleaning tank;
A chemical dispensing system (optional) for dosing chemicals to the liquid supplied to the cleaning tank;
A steam generator for providing steam to the cleaning tank for cleaning lumens (optional);
Electrical power controllers and switches and electrical supply for the cleaning tank and the rinsing tank. In addition, a power controller is adapted to set and/or determine the frequency for the ultrasound transducers of the cleaning tank, e.g. to determine the basic frequency and the modulation frequency as well as the power supplied to the ultrasonic transducers.
A sensor system for sensing cleaning liquid temperature, sensing liquid levels of cleaning liquid in the cleaning tank and alarms systems and also the same sensor system for the rinsing tank optionally.

### BRIEF DESCRIPTION OF THE DRAWINGS

FIG. 1 is a top view of an ultrasonic cleaning tank according to an embodiment of the present invention.

FIG. 2 is an end view of the ultrasonic cleaning tank of FIG. 1.

FIG. 3 is a side view of the ultrasonic cleaning tank of FIG. 1.

FIG. 4 is a bottom view of the ultrasonic cleaning tank of FIG. 1

FIG. 5 is a cross-section through the ultrasonic cleaning tank of FIG. 1 along the section A-A.

FIG. 6a and FIG. 6b are views of a rinsing tank according to an embodiment of the present invention.

FIG. 7 is a schematic representation of a control system for a console cleaning system according to an embodiment of the present invention.

### DETAILED DESCRIPTION OF THE INVENTION

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

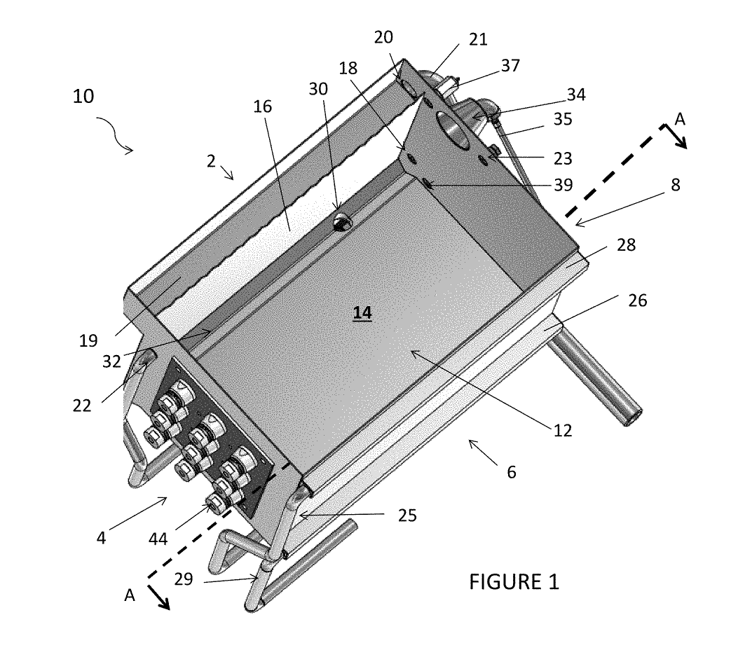

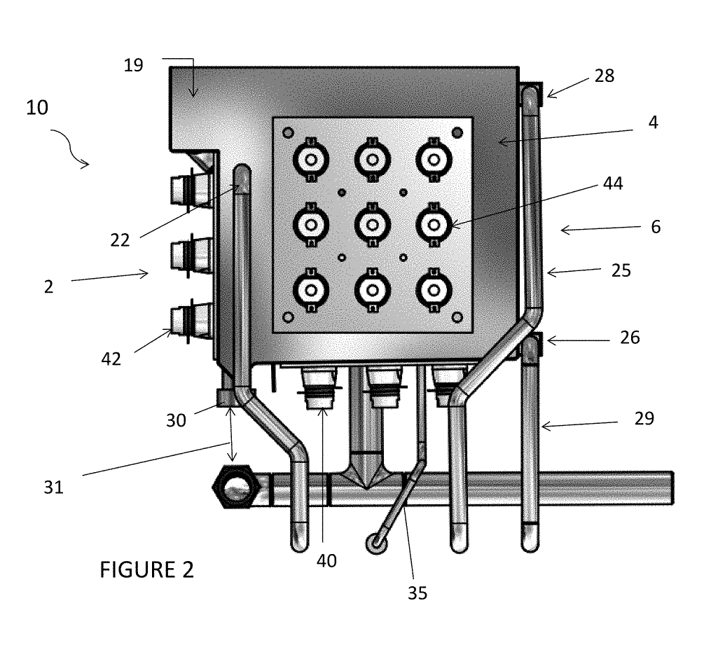

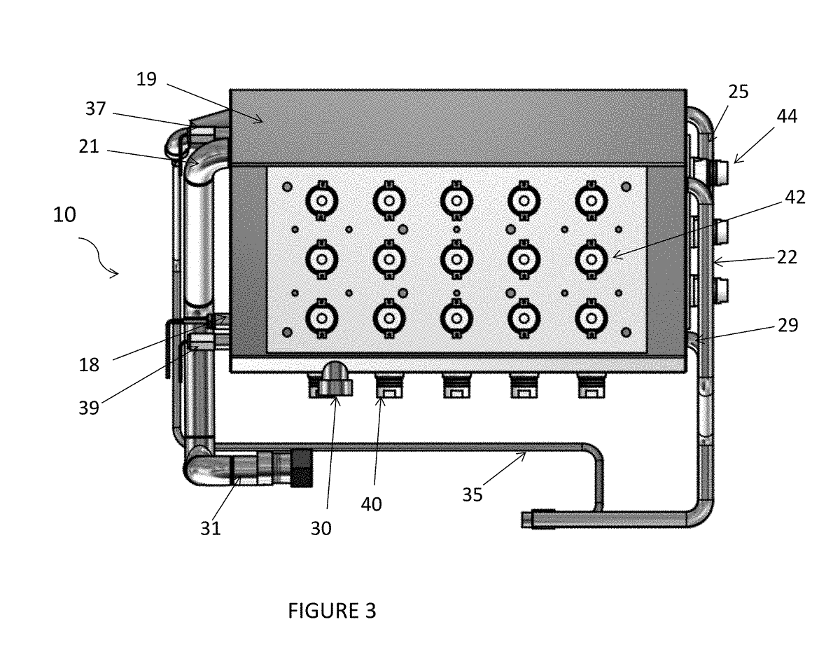

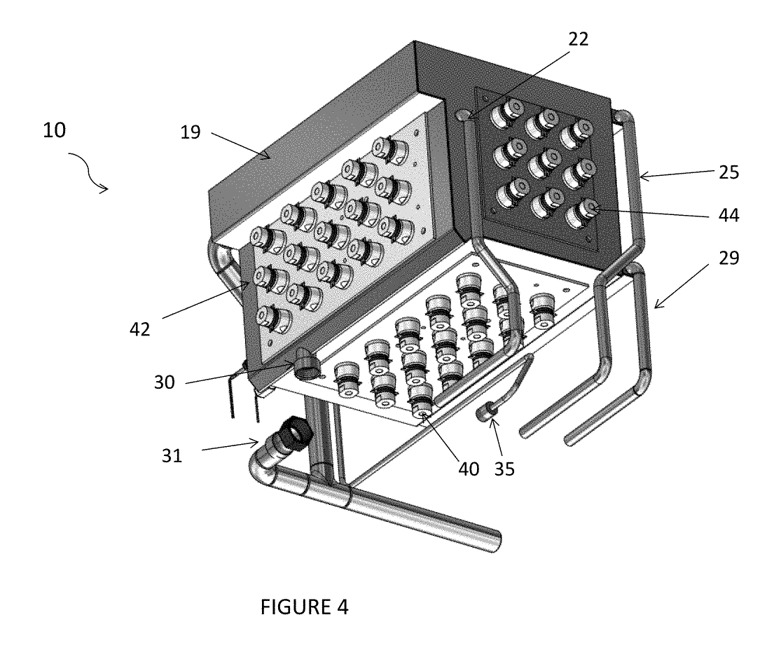

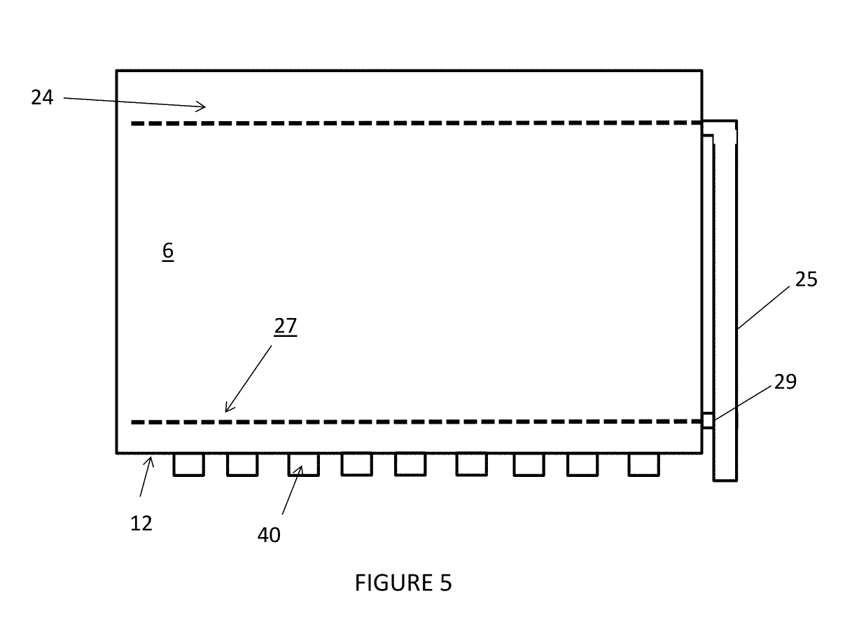

As shown schematically in FIGS. 1 to 5, a bath, tub, basin or sink 10 of an embodiment of the present invention includes one or more walls 2, 4, 6, 8, and a bottom 12 defining a tank volume 14 for holding a liquid for pre-cleaning or cleaning of contaminated objects such as used medical and/or surgical devices. In the description and claims the term “tank” will be understood to include any of a bath, tub, basin or sink. The form and size of the tank 10 can be chosen based on the size of the objects to be cleaned. Some of these objects can have an open hollow part such as a lumen. The preferred liquid used for cleaning is water with or without other chemicals. Other aqueous solutions that can be used can include enzymatic solutions or solutions including detergents, soaps, antiseptics, disinfectants, silver based ionic species, etc. Any cleaning chemicals or biologicals can be introduced into tank 10 through port 23. In some cleaning processes other liquids can be used such as isopropyl alcohol, ethanol, acetone or mixtures of these or any other suitable solvent cleaning chemicals can be used as the cleaning liquid.

The tank 10 is preferably held in a frame (not shown), preferably at an ergonomic working height. The tank 10 is adapted to carry out one or more cleaning procedures, e.g. of soaking, scrubbing, ultrasonic cleaning, ultrasonic cleaning with or without cavitation, rinsing, pre-cleaning. Preferably the tank and the cleaning process are brushless, i.e. brushing is not used as brushes can retain debris or detritus and are therefore difficult to clean.

In an embodiment the tank 10 has a plurality of walls (e.g. four 2, 4, 6, 8) extending upwardly from the bottom 12 of the tank 10 to enclose a tank volume 14. The walls (e.g. 2, 4, 6, 8) can be flat but also curved walls could be used, e.g. forming a circular, elliptical or oval shape in horizontal cross-section. The tank 10 can have, for example one outer circular wall, and an inner circular wall whereby either the inner wall or the outer wall forms a weir. In another example, the tank 10 can be an elliptical tank having two half-elliptical walls and a bottom. The tank 10 should preferably have at least two walls facing each other and enclosing a volume 14. For example, the tank 10 can have four walls 2, 4, 6, 8 (two side walls 2 and 6 and two end walls 4 and 8) forming a convenient shape such as square or rectangular. Other shapes may be suitable depending upon the application.

The wall or walls 2, 4, 6, 8 and bottom 12 should be smooth and should have no blind spots or places where eddies or dead spaces can form. The material from which the walls 2, 4, 6, 8 and bottom 12 are made should not support attached cell growth, e.g. of human cells, bacteria, algae, amoebas, microorganisms, parasites, plants, or other biological materials. Suitable properties for the material of the tank 10 are strength and rigidity, chemical and temperature resistance, good impact, abrasion, cracking and shatter resistance, surface hardness, and chemical inertness. The bottom 12 can be horizontal, i.e. is flat and horizontal or may slope downwards towards the second channel 32—see below, i.e. be flat and sloping.

For cleaning medical devices, the material of the walls 2, 4, 6, 8 or bottom 12 should also not initiate coagulation of proteins. For cleaning medical devices the tank 10 should be operated at a temperature of less than 60° C., less than 45° C. and preferably in the range of 30 to 40° C. to avoid coagulation of proteins.

A preferred material for the tank 10 is stainless steel. Alternatively, another appropriate metal could be selected for the material of the body of the tank 10. If the tank 10 is made as a replaceable insert then other materials, such as plastic, can be suitable. Such a material can be a synthetic resin. The tank 10 can be made by any suitable method such as machining, fabrication and welding, or moulding from a plastic material. Low surface energy non-polar plastics are preferred such as polypropylene or polyethylene.

The upper edges of the tank 10 are preferably continuous, smooth and rounded so that there is no risk of injury to operators. The upper edges can be designed as flanges such that if the tank 10 is constructed as an insert, then the insert can hang from these edges when lowered into a frame (not shown).

The tank 10 preferably has one or more weirs 16 that allow overflow of the liquid from the tank volume 14 of the tank 10. The one or more weirs 16 are spaced from, i.e. opposite or parallel to a wall 6 of the tank 10, to form thereby a first channel 19. The weir 16 may be parallel to the wall 6 of the tank. On the side of the one or more weirs 16 which is remote from the main tank volume 14 of the tank 10, one or more drains 20 are provided which communicate with a first channel 19 to drain liquid and debris or detritus from the first channel 19. The tank 10 is preferably adapted to make liquid flow over the weir 16, by injecting cleaning liquid into tank 10 through an inlet 22 and allowing the cleaning liquid to flow until it reaches the one or more drains 20 with drain conduits 21, which flow assists in moving floating debris or detritus to the one or more drains 20. This flow over the weir 16 may be arranged to be continuous, intermittent or pulsating depending on the provision and configuration of a liquid feed, pumps and valves. To push floating debris and detritus towards the weir 16 a skimmer 28 is provided. This skimmer 28 can be a plurality of holes or nozzles 24 which are provided in a wall 6 of the tank 10 in a line, opposite to the weir 16 and at the upper level of the liquid in the tank 10. Liquid and/or gas can be pumped from a conduit 25 into the tank volume 14 through the holes or nozzles 24 to drive floating debris and detritus towards and over the weir 16 and, hence, to the one or more drains 20. The skimmer 28 preferably extends for the complete length of the wall 6 opposite the weir 16 so that the flow of liquid out of the holes or nozzles 24 does not generate eddies where material can become trapped but rather produces a uniform movement across the tank volume 14 of the liquid. The nozzles 24 can be in the form of holes or a continuous slit or slits. The holes should be close enough together to avoid eddies being formed between the flow from one hole and an adjacent flow from the adjacent hole. If necessary more than one row of holes or more than one slit can be provided. The weir 16 is in a topological relationship to the wall 6 such that a surface liquid flow initiated at the wall 6 traverses the complete surface of the cleaning liquid and reaches the weir 16, whereby each part of the surface flow initiated at the wall 6 maps to a part of the weir 16 such that no dead spaces are created within the surface flow. The flow is preferably of a laminar nature.

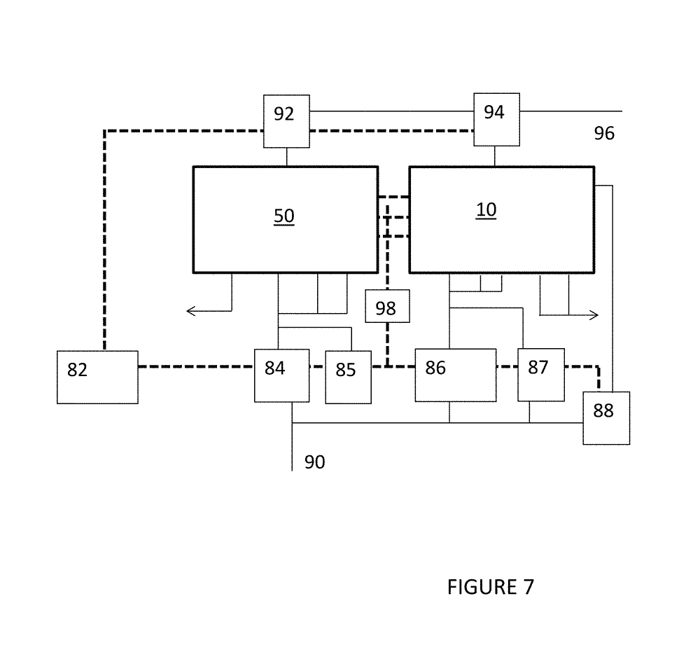

An upper liquid level sensor 37 is provided to allow control of the height of the upper surface of the liquid so that there is sufficient flow over the weir 16. Pumps and valves and a controller are provided to regulate the level of the liquid in the tank 10, e.g. based on the signals from the upper liquid level sensor 37 and a lower level sensor 39 (see FIG. 7). The top edge of the weir 16 may be flat or may have a series of teeth, i.e. be serrated. The flow through the one or more drains 20 may be due to gravity, or suction may be applied to the drain 20 to suck out liquid, debris and detritus, e.g. by provision of a suitable pump. In addition or alternatively, a barrier plate or swimmer (not shown) may be physically moved across the top of the liquid in the tank 10, e.g. by means of hydraulic rams to push the detritus or debris towards and over the weir 16 and, hence, to the one or more drains 20. A plate whether solid or porous in the form of a mesh is however less preferred in comparison to the holes or nozzles 24, as a plate may be more difficult to keep clean.

Debris and detritus that does not float will sink to the bottom 12 where one or more further drains 30 are provided. The bottom 12 of the tank 10 preferably has a channel 32 in which the one or more further drains 30 are located connected to drain conduits 31. The channel 32 extends along the bottom 12 and allows debris and detritus to be collected and to be moved towards the one or more further drains 30. Preferably, the channel 32 extends along the complete length of the tank 10 so that there are no flat surfaces where eddies can form and detritus or debris is deposited. It is preferred if the channel 32 has a V form with the apex facing down. The tank 10 is adapted so that flow of liquid from an inlet 22 of the tank 10 to the one or more further drains 30 causes a flow of liquid along the channel 32 and this flow assists in moving debris or detritus along the channel 32 to the one or more further drains 30. This flow of liquid towards and through the channel 32 can be made continuous, intermittent or pulsating depending on the provision and configuration of a liquid feed, pumps and valves. The flow through the one or more further drains 30 may be due to gravity or suction may be applied to the one or more further drains to suck out liquid, debris and detritus, e.g. by use of a suitable pump. To assist in this further draining a further skimmer 26 is provided. This further skimmer 26 can be a plurality of holes or nozzles 27 or a slit or slits which are provided in a line in a wall 6 of the tank 10 opposite to the one or more further drains 30 and at the level near to or at the bottom 12 of the tank 10. Liquid can be pumped through this line of holes or nozzles 27 or slit or slits from a conduit 29 to drive debris and detritus lying on the bottom 12 towards the one or more further drains 30. In addition or alternatively, a barrier plate may be physically moved over the bottom 14 of the tank 10, e.g. by means of hydraulic rams to push the detritus or debris towards the drain channel 32 and the one or more further drains 30. The bottom of the plate may comprise a rubber squeegee. A plate is however less preferred in comparison to holes or nozzles 27, as a plate may be more difficult to keep clean.

In this aspect of the present invention the second channel has a topological relationship to the at least one wall and/or the weir and/or at least one second wall such that a second liquid flow initiated at the level of the bottom 12 of the tank 10 traverses the complete bottom of the tank and reaches the second channel 32 and drain 30, whereby each part of the bottom flow maps to a part of the second channel 32 such that no dead spaces are created within the bottom flow. The flow is preferably of a laminar nature. A quiet flow is preferred because excessive turbulence may lift soils from the bottom and re-attach them to the medical and/or surgical devices to be cleaned.

The tank 10 is provided with sufficient electrical supplies, pumps, conduits and valves and access to drains to allow continuous, pulsating or intermittent flow of liquid through the tank 10, and through the first and/or second skimmers 28 and 26. Also an outlet 34 such as a nozzle, liquid supply and a pump is provided for jetting liquid and/or steam and/or solvents optionally at high pressure through the outlet 34 as supplied through conduit 35 to flush hollow spaces such as a lumen of a medical or surgical device. This cleaning is preferably done by hand as the control of the cleaning process can then be extended or modified by human intervention. The jetting can be continuous, pulsating or intermittent. The outlet 34 may include a nozzle and/or may include a tube extension or a flexible tube extension (not shown) that allows the jetted liquid and/or steam to be directed into a lumen of a medical or surgical device. The end of any such tube or outlet 34 may be fitted with a means for attachment to the lumen of the medical or surgical device to be cleaned. Such means can be an adaptor which can be constructed for sealed attachment both to the outlet or outlet tube as well as to the lumen.

A control unit is provided which can be programmed to operate the pumps and valves in a manner suitable to control the cleaning process (see FIG. 7). Such a controller may be a PLC which has been suitably programmed.

Various supports or other devices to accommodate or to hold objects to be cleaned such as hooks, racks, baskets, trays or caddies can be provided and are not considered to be a limitation on the present invention. It is preferred if the racks, baskets, trays and caddies are used to hold medical or surgical devices even if these devices are small in size and, hence, to avoid the need for a filter to prevent small devices to leave through the one or more further drains 30. In other words, any openings in the racks, baskets, trays and caddies should be dimensioned to hold back any expected medical and surgical devices. Optionally, but less preferred, catch elements of a filter can be provided which are shaped and located to hold back small medical and surgical devices or other small objects to remove such objects before the liquid drains out through the one or more drains 20 or 30.

A measurement system is preferably provided to measure any or some or all of:
a) The amount of any additives to be added to the cleaning liquid. Additives can be provided from any suitable source and introduced to tank 10 through a port 23.
b) Level of the liquid in the tank volume 14 by using signals from upper 37 and lower 39 liquid level sensors.
c) Temperature of the liquid by means of a temperature sensor 18.
d) Any restrictions on liquid flow which are indicative of blockages, e.g. using signals from flow measurement devices (not shown) in any of the conduits or drains.

Any, some or all of these may be logged automatically to demonstrate that the cleaning process was carried out correctly. Any, some or all of these may be used as input for a control system.

The tank 10 is used with an ultrasonic generator for generating ultrasonic energy such as a solid state ultrasonic generator. The output of the generator can be a square wave, slowly or rapidly pulses of ultrasonic energy, modulating or sweeping the frequency of the generator output around the central operating frequency, e.g. within the range of 40 kHz to more than 1 MHz such as up to 10 MHz. Such an ultrasonic generator can be adapted to adjust a variety of output parameters and to customize the ultrasonic energy output for the application.

The tank 10 is provided with ultrasound transducers 40, 42, 44 for subjecting the cleaning liquid in the tank volume 14 and, hence, also the medical or surgical devices to ultrasound. The ultrasound transducers are arranged on the outside of the tank 10 and in particular on the bottom 12 (ultrasound transducers 40) and on one of the walls 2, 4, 6, 8, (ultrasound transducers 42 or 44), more preferably on the bottom 12 (ultrasound transducers 40) and on two of the walls (for example on walls 2, 4, ultrasound transducers 42 and 44), and are preferably aligned such that the ultrasound is emitted essentially from the walls and bottom into the tank volume 14 of the liquid. The ultrasound transducers can be arranged on the bottom 12 and on three or on all four walls 2, 4, 6, 8 (not shown). The ultrasound energy inserted into the tank volume 14 of the liquid enters in different directions which helps to prevent ultrasound shadows. The ultrasound energy inserted into the tank volume 14 of the liquid from all of the transducers is in the range 20 to 40 watts per litre, e.g. 30 to 35 watts per litre. In case this energy density overheats the cleaning liquid a cooling stage can be included in the conduits for liquid supply which is automatically controlled by the controller and controls the temperature of the liquid which is circulated through the tank volume 14.

Alternatively, immersible ultrasonic transducers can be mounted on the sides or bottom of the cleaning tank.

The ultrasound transducers can be, for example, piezo electric sound-generators or other ultrasound producers which are known from the state of the art. The ultrasound transducers can be arranged, for example, along several rows. Alternatively, magnetostrictive transducers may be used.

Applying a square wave signal to the ultrasonic transducers results in an acoustic output having many harmonics. This provides a multi-frequency cleaning system which operates simultaneously at several frequencies which are harmonics of the fundamental frequency. Multi-frequency operation offers the benefits of all frequencies combined in a single ultrasonic cleaning tank.

In pulsed operation, the ultrasonic energy is turned on and off at a rate which may vary from once every several seconds to several hundred times per second.

The duty cycle of the ultrasonic energy can be varied to adapt the energy generation to specific applications. Low pulse rates can assist in rapid degassing of liquids whereas at higher pulse rates the cleaning liquid is subject to repeated high energy bursts of ultrasonic energy. The ultrasonic transducers may be fired simultaneously or sequentially.

It is preferred that the frequency of the output of the ultrasonic generator is modulated around a central frequency which itself can be adjustable. The frequency and amplitude of the frequency modulation can be adjusted. A suitable frequency range is from 40 kHz to above 1 MHz, e.g. up to 10 MHz.

Various methods can be combined such as a combination of pulsed and sweep operation. In the ultrasonic cleaning process various process parameters can be altered such as time, temperature and the use of chemicals, and intensity of ultrasonic cavitation in the liquid. Cavitation of the cleaning liquid can be affected by temperature and any of the embodiments of the present invention can have heaters and coolers, temperature sensors in the cleaning tank and controllers therefor. The temperature is preferably limited to less than 60° C., less than 45° C. and preferably in the range 30 to 40° C. to avoid coagulation of proteins.

Vapourous cavitation, in which the cavitation bubbles are filled with the vapour of the cavitating liquid can also be used with embodiments of the present invention which requires degassing of the cleaning liquid before use. Degassing is done preferably after any chemical is added and can be accomplished in the tank by operating with ultrasonic energy and raising the solution temperature to the acceptable limit, e.g. less than 60° C., less than 45° C. and preferably in the range 30 to 40° C.

Cavitation intensity can be directly related to the ultrasonic power. Cavitation intensity is inversely related to ultrasonic frequency. As the ultrasonic frequency is increased, cavitation intensity is reduced because of the smaller size of the cavitation bubbles and their resultant less violent implosion. Embodiments of the present invention overcome the reduction in cavitation effect at higher frequencies by increasing the ultrasonic power.

In another embodiment a console cleaning system is provided that integrates one or more ultrasonic cleaning tanks as described above with reference to FIGS. 1 to 5, with other tanks such as rinse tanks, or washing machines or a dryer for batch cleaning. In accordance with another aspect of the present invention such a system is automated through the use of a PLC.

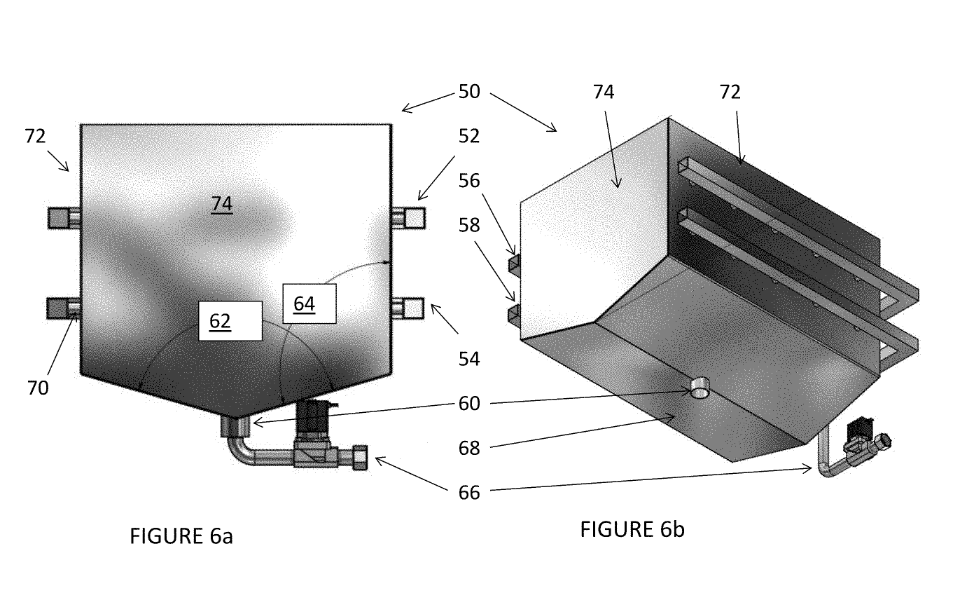

The console cleaning system can include one or more ultrasonic cleaning tanks 10 as described above with reference to FIGS. 1 to 5, and a rinsing tank 50 as shown in FIGS. 6a and 6b. This rinsing tank 50 is also preferably brushless. In the console the rinsing tank 50 is placed first followed by one or more ultrasonic cleaning tanks 10.

The rinsing tank 50 has a V shaped bottom 68 with a drain 60 at its lowest point. The bottom plates of the V shape are preferably at an angle 62, 64 steep enough which allows debris and detritus to be washed down to the drain 60. The bottom 68 can also be shaped as any suitable depression such as a pyramidal or bowl shaped with the drain 60 located at the lowest point. Suitable properties for the rinsing tank 50 are strength and rigidity, chemical and temperature resistance, good impact, abrasion, cracking and shatter resistance, surface hardness, and chemical inertness. A preferred material for the rinsing tank 50 is stainless steel. Alternatively, another appropriate metal could be selected for the material of the body of the rinsing tank 50. If the rinsing tank 50 is made as a replaceable insert then other materials such as plastic can be suitable. Such a material can be a synthetic resin. The rinsing tank 50 can be made by any suitable method such as machining, fabrication and welding, or moulding from a plastic material. Low surface energy non-polar plastics are preferred such as polypropylene or polyethylene.

The upper part of the rinsing tank 50 can be straight vertical side walls 72 and end walls 74. At least one of the side walls 72 and preferably both side walls 72 (as shown) are provided with nozzles 70 in one or more rows, preferably two rows (as shown) which are fed with liquid such as water and/or steam at high pressure along conduits 52, 54, 56, 58 from a supply line 66 controlled by suitable valves and a valve controller. End walls 74 can also be provided with nozzles 70. A nozzle with flexible tubing (not shown) may be provided for rinsing medical or surgical devices with a lumen. The nozzles 70 may also optionally provide compressed air. The nozzles 70 provide a spray of liquid into the rinsing tank 50.

The cleaning tank 10 and the rinsing tank 50 may be located in suitable furniture such as a table with connections for electrical power, liquid, steam, air pressure and chemical supply.

FIG. 7 is a schematic representation of a control system for a console cleaning system according to an embodiment of the present invention. A main controller 82 such as a PLC controls of the operation of the following optional control elements:
A source 90 of cleaning liquid;
A pump and valve system 84 to provide liquid to rinsing tank 50;
A chemical dispensing system 85 for dosing chemicals (optional) to the liquid supplied to the rinsing tank 50;
A pump and valve system 86 to provide liquid to cleaning tank 10;
A chemical dispensing system 87 (optional) for dosing chemicals to the liquid supplied to the cleaning tank 10 through port 23;
A steam generator 88 for providing steam to the cleaning tank 10 for cleaning lumens (optional) through the port 34;
Electrical power controllers and switches 92 and 94 and electrical supply 96 for the rinsing tank 50 and the cleaning tank 10. In addition, a power controller 92 is adapted to set/determine the frequency for the ultrasound transducers of the cleaning tank 10, e.g. to determine the basic frequency and the modulation frequency as well as the power supplied to the transducers, as well as continuous pulsed, or sequences operation of the ultrasonic transducers.
Sensor system 98 for sensing the cleaning liquid temperature from sensor 18, sensing levels of cleaning liquid in the cleaning tank 10 with upper sensor 37 and lower sensor 39 and alarm systems and also the same sensor system 98 for the rinsing tank 50 optionally.

The individual elements can be used to drive the rinsing tank 50 or the cleaning tank 10 separately.

1. Apparatus for ultrasonic pre-cleaning or cleaning of objects such as medical or surgical devices optionally having a lumen, the apparatus comprising:
a tank having a tank volume for holding cleaning liquid, the tank having at least one first wall and a bottom and being adapted to hold cleaning liquid and to expose the cleaning liquid to ultrasound when in the tank;
a weir facing the at least one first wall,
a first channel adjacent and proximal to the weir and distal with respect to the tank volume;
a first drain located in said first channel for draining cleaning liquid that spills over the weir;
a second channel in the form of a depression in the bottom of the tank, and
a second drain located in the second channel for draining cleaning liquid from the tank.

2. The apparatus according to claim 1 comprising ultrasonic transducers for inserting ultrasonic energy through the bottom of the tank and/or through the at least one first wall and/or through other walls.

3. The apparatus according to claim 1 further comprising an outlet for delivering cleaning liquid and/or steam for the cleaning of objects with a lumen.

4. The apparatus according to claim 1, further comprising a first skimmer, for moving floating debris or detritus towards and over the weir.

5. The apparatus according to claim 4, wherein the first skimmer is adapted to insert cleaning liquid through the at least one first wall and for moving floating debris or detritus towards and over the weir.

6. The apparatus according to claim 5, wherein the first skimmer comprises a first line of holes located in the at least one first wall for inserting liquid through the at least one first wall.

7. The apparatus according to claim 4, wherein the weir is in a topological relationship to the at least one wall such that a surface liquid flow initiated at the at least one wall traverses the complete surface of the cleaning liquid and reaches the weir, whereby each part of the surface liquid flow initiated at the at least one wall maps to a part of the weir such that no dead spaces are created within the surface liquid flow.

8. The apparatus according to claim 1, further comprising a second skimmer for moving sunken debris or detritus along the bottom of the tank towards the second channel and down the second drain.

9. The apparatus according to claim 8, wherein the second skimmer is adapted to insert cleaning liquid through the at least one first wall for moving sunken debris or detritus towards the second channel and down the second drain.

10. The apparatus according to claim 9, wherein the second skimmer comprises a second line of holes located in the at least one first wall and for inserting liquid through the at least one first wall.

11. The apparatus of claim 8, wherein the second channel has a topological relationship to the at least one wall and/or the weir such that a bottom liquid flow initiated at the level of the bottom of the tank traverses the complete bottom of the tank and reaches the second channel and drain, whereby each part of the bottom liquid flow maps to a part of the second channel such that no dead spaces are created within the bottom liquid flow.

12. The apparatus according to claim 1, wherein the cleaning liquid is exposed to 20 to 40 watts per litre, or 30 to 35 watts per litre of ultrasound energy.

13. The apparatus according to claim 1, wherein the apparatus is brushless.

14. A method of ultrasonic pre-cleaning or cleaning of objects such as medical or surgical devices optionally having a lumen, in a tank holding cleaning liquid, the tank having at least one first wall and a bottom and being adapted to hold cleaning liquid and to expose the cleaning liquid to ultrasound;
a weir opposite to the at least one first wall and a first channel adjacent and proximal to the weir and distal with respect to the tank; and
ultrasonic transponders for injecting ultrasonic energy into the tank,
the method comprising:
removing floating detritus or debris by a first directing of cleaning liquid to spill over the weir and draining the floating detritus or debris and cleaning liquid from the tank, and
removing sunken detritus or debris by a second directing of cleaning liquid to flow along the bottom of the tank and draining the sunken detritus or debris and cleaning liquid from the tank.

15. The method of claim 14, wherein the first and/or second directing step includes injecting cleaning liquid into the tank through the at least one first wall.

16. The method according to claim 14, further comprising exposing the cleaning liquid to 20 to 40 watts per litre, or 30 to 35 watts per litre of ultrasound energy.

17. The method of claim 14, further comprising the step of delivering cleaning liquid and/or steam for the cleaning of objects with a lumen through an outlet.

18. A console pre-cleaning or cleaning system comprising a cleaning tank and a rinsing tank, the cleaning tank being an apparatus for ultrasonic pre-cleaning or cleaning of objects such as medical or surgical devices optionally having a lumen, the apparatus comprising:
a tank having a tank volume for holding cleaning liquid, the tank having at least one first wall and a bottom and being adapted to hold cleaning liquid and to expose the cleaning liquid to ultrasound when in the tank;
a weir facing the at least one first wall,
a first channel adjacent and proximal to the weir and distal with respect to the tank volume;
a first drain located in said first channel for draining cleaning liquid that spills over the weir;
a second channel in the form of a depression in the bottom of the tank, and
a second drain located in the second channel for draining cleaning liquid from the tank,
the rinsing tank having a depressed bottom with a drain at the lowest point and one or more rows of nozzles for delivery into the rinsing tank of a spray of cleaning liquid and/or steam and/or compressed air.

19. The console pre-cleaning or cleaning system according to claim 18, further comprising:
a first skimmer, for moving floating debris or detritus towards and over the weir, the first skimmer being adapted to insert cleaning liquid through the at least one first wall and for moving floating debris or detritus towards and over the weir and a second skimmer for moving sunken debris or detritus along the bottom of the tank towards the second channel and down the second drain.

20. The console pre-cleaning or cleaning system according to claim 18 further comprising
a source of cleaning liquid;
a pump and a valve system to provide liquid to the rinsing tank;
a chemical dispensing system for dosing chemicals to the liquid supplied to the rinsing tank;
a pump and valve system to provide liquid to cleaning tank;
a chemical dispensing system for dosing chemicals to the liquid supplied to the cleaning tank;
a steam generator for providing steam to the cleaning tank for cleaning lumens;
electrical power controllers and switches and electrical supply for the rinsing tank and the cleaning tank;
a power controller adapted to set/determine the frequency for the ultrasound transducers of the cleaning tank;
a power controller adapted to determine the basic frequency and the modulation frequency as well as the power supplied to the transducers, as well as continuous pulsed, or sequences operation of the ultrasonic transducers;
a sensor system for sensing the cleaning liquid temperature, sensing levels of cleaning liquid in the cleaning tank and an alarm system.
